(19) United States
(12) Patent Application Publication
(10) Pub. No.: US 2026/0117151 A1
(43) Pub. Date: Apr. 30, 2026

(54) COMPOSITION FOR THE PREPARATION OF ALCOHOLIC BEVERAGES BASED ON Heliopsis longipes AND ITS MANUFACTURING PROCESS

(71) Applicant: Mariana Torres Landa Aranda, Guanajuato (MX)

(72) Inventor: Mariana Torres Landa Aranda, Guanajuato (MX)

(21) Appl. No.: 19/372,360

(22) Filed: Oct. 29, 2025

(30) Foreign Application Priority Data
Oct. 30, 2024 (MX) MX/A/2024/013406

(51) Int. Cl. C12G 3/055; A61K 36/28

(52) U.S. Cl. CPC C12G 3/055; A61K 36/28; A61K 2236/13; A61K 2236/15; A61K 2236/17; A61K 2236/333

(57) ABSTRACT

A formulation made from Heliopsis longipes, a perennial plant commonly known as chilcuague. The plant presents a high concentration of bioactive compounds in its root, which can be effectively utilized when extracted using appropriate solvents such as ethanol. In contrast, aqueous infusions yield low detectable levels of such compounds. The extraction process significantly influences the physicochemical parameters of the resulting solution, particularly its color and, to a lesser extent, its pH. This provides an opportunity to develop food products that are regularly consumed while incorporating high levels of bioactive extracts into their formulations. The process includes the steps of immersion cleaning, hydration, bark tearing, maceration, and preservation—Heliopsis longipes root transfers its organoleptic and functional properties to an alcoholic vector, producing an innovative composition for the elaboration of alcoholic beverages such as tequila, rum, vodka, gin, and others.

### FIELD OF THE INVENTION

The present invention relates to the preparation of a formulation made from Heliopsis longipes, a perennial plant commonly known as “chilcuague,” which is endemic to the Sierra de Álvarez and Sierra Gorda in Mexico—regions spanning parts of the states of San Luis Potosí, Guanajuato, and Queretaro—whose root is known to have traditional uses. Among the disclosures concerning the plant, the use of chilcuague as a deworming agent is recognized, by adding the roots to foods. In view of its characteristics, the present invention is regarded as a chemical patent.

### BACKGROUND OF THE INVENTION

Plants have long been used to prepare alcoholic beverages from agaves and cereals such as barley, rye, and corn, which have a high starch content whose hydrolysis yields the sugars needed for alcoholic fermentation.

These alcoholic beverages prepared by such processes are referred to as “white spirits,” including, among others, tequila, rum, vodka, gin, cachaça, pisco, etc. They are characterized by an alcohol content above 30% v/v ethanol and, although all are obtained by fractional distillation of fermented liquids, differences in their manufacture yield the subsequent distinctions that characterize each beverage.

From an analytical standpoint, gas chromatography is one of the most commonly used methods to quantify the ethanol content in these beverages and to corroborate the nominal ethanol content stated on the label. However, there are no analytical methods capable of reliably distinguishing each of these beverages. This opens an opportunity to generate compositions that first modify organoleptic characteristics and, thanks to plant properties, allow compositions that enhance certain technical effects such as antiseptic, astringent, and antiparasitic activity, among others.

On the other hand, official standards or regulations imposed on beverages protected by denominations of origin or, where applicable, certifications and geographical indications, prevent alteration of the standards required for a beverage to be considered as defined by law.

Conventional extracts used to modify the organoleptic characteristics of these beverages are typically citrus, berries, barks such as cinnamon, ginger, and the like.

The present invention introduces the use of “Chicuagüe” (chilcuague). Within the genus, the species Heliopsis longipes has the greatest economic importance (Fisher, 1954). Its recognized insecticidal and medicinal qualities include using its root as a seasoning in sauces, stews, and alcoholic beverages (Martínez, 1967). In traditional medicine it is used to relieve muscle and tooth pain, as well as a deworming agent and insecticide (Little, 1948b; Martínez, 1967).

The use of chilcuague has involved destroying the entire plant; as a result, populations of this species have declined and disappeared in some areas (Little, 1948b; Molina-Torres and Garcia-Chávez, 2001). Chemical (Jacobson, 1954; Jacobson, 1955; Domínguez et al., 1958; Molina-Torres et al., 1995; Molina-Torres et al., 1996; García-Chávez et al., 2004) and pharmacological (Johns et al., 1982; Romero et al., 1989; Gutiérrez-Lugo et al., 1996; Molina-Torres et al., 1999; Ramírez-Chávez et al., 2000) studies have been conducted on the root of H. longipes.

Its pungent taste and the numbing sensation it produces lead to its combination with chili or use as a substitute. It relieves toothache when held against the affected tooth; for muscle pain it is used macerated in alcohol; as a deworming agent, a piece of root is chewed or consumed in foods.

Although traditionally present in preparations related to certain customary uses, the plant has been only incipiently studied due to the reduction in its occurrence and the scarcity of this endemic species.

Among the ailments associated with its traditional uses are cold sores, muscle pain, arthritis, rheumatism, diarrhea, deworming, gum-related problems, oral infections, toothache, anti-inflammatory effects, and, in culinary applications, as a seasoning; it is also used as a traditional remedy against so-called “empacho.”

### PRIOR ART

In the state of the art, several patents disclose the use of Heliopsis longipes in various compositions, as described below:

The patent WO2017010858A1, entitled “Composition based on extracts of Heliopsis longipes and propolis for the control and/or elimination of ticks,” refers to a natural extract-based insecticidal composition for controlling and/or eliminating ticks in livestock and/or domestic animals. It is characterized in that it comprises: (a) 50% to 65% of Heliopsis longipes extract; (b) 5% to 20% of propolis extract; and (c) 30% of a dermal vehicle, all adding up to 100%.

The mixture of the composition presents a synergistic effect ensuring control of adult ticks, nymphs, pupae, larvae, and eggs—particularly in adult females, altering their oviposition capacity and affecting egg hatching and larval survival. This includes all ticks of veterinary importance from the Ixodidae and Argasidae families.

This patent claims a composition based on natural extracts for the control and/or elimination of ticks, characterized by: (a) a natural extract of Heliopsis longipes; (b) a natural extract of propolis; and (c) a dermal vehicle—thus supporting its antiparasitic effect but not as a preparation for alcoholic beverages.

The patent MX 251313 B, entitled “Herbal composition to improve oral hygiene and methods of using same,” comprises formulations based on Heliopsis longipes root used orally to treat xerostomia, dry mouth, to increase salivation, to relieve dry mouth, and to maintain or improve oral hygiene. It also acts as a topical anesthetic by contact, a cough suppressant, and an oral sensitizer producing desirable sensations in the oropharynx.

The herbal composition includes a Heliopsis longipes root extract and an oral vehicle for the extract. An adequate amount of extract ranges from approximately 0.01% to about 10% by weight of the total composition. Various compositions include an oral vehicle; the preferred ones are: (1) chewing gum with a solid outer shell and liquid core, both containing an effective amount of extract composition; (2) solid gum; (3) lozenges, candies, and confections; and (4) flavored sprays, drops, and mouth rinses. The invention is also useful as a flavor enhancer and taste booster.

Another application, MX/a/2022/011183, entitled “Use of ethanolic extract of Heliopsis longipes and affinin, its major alkamide, for treating arterial hypertension,” seeks to protect the method for obtaining the ethanolic extract of Heliopsis longipes roots and for purifying affinin, its major alkamide, as well as its use as an antihypertensive agent for treating cardiovascular diseases, including hypertension.

The antihypertensive effect of affinin involves a reduction in endothelial dysfunction and an increase in nitric oxide levels. Both the ethanolic extract and affinin induce an antihypertensive effect similar to that of captopril, one of the first-line drugs in hypertension treatment.

A relevant process disclosed in that application and considered a background for the present invention is the maceration in alcohol, described as follows:

Method for preparing the ethanolic extract of Heliopsis longipes root (EEHI):

Dry the roots of Heliopsis longipes in air at room temperature, in a dark, well-ventilated room for two weeks.

Grind the dried roots of Heliopsis longipes into a fine powder.

Perform maceration with a polar solvent, preferably absolute ethanol, at a ratio of 1:10 (m/v), for a period of 6 to 10 days, preferably for 7 days.

Repeat this process three times with fresh solvent.

Filter under vacuum to remove plant material.

Evaporate the solvent under reduced pressure using a rotary evaporator to obtain the dry extract (EEHI), which is a poorly water-soluble, reddish-brown, oily product.

As a variant, the patent discloses a similar method for preparing an ethanolic extract of Heliopsis longipes root (EEHI), according to the description above, characterized in that the resulting product is a viscous oil, reddish-brown in color, and poorly soluble in water.

The patent US20170143022A1, entitled “Compositions incorporating a flavoring agent for umami taste,” describes formulations containing taste modifiers, such as flavoring or taste-enhancing agents—particularly modifiers of salty taste (umami), savory flavoring agents, and taste enhancers for food, beverages, and other edible compositions. The described compositions include an umami flavoring or taste-enhancing agent in combination with one or more additional food additives, preferably flavoring substances, plants, spices, fats, or oils. This patent, however, is directed toward flavor modification in food, not alcoholic beverages.

The patent RU2497503C9, entitled “Solid composition of pleasant taste containing a deacidifying and salivation-stimulating agent,” relates to the pharmaceutical industry. It represents a solid composition with a pleasant taste for treating or preventing gastric discomfort or diseases such as acid indigestion, heartburn, or gastritis, containing at least one deacidifying and neutral salivation stimulant.

The neutral salivation stimulant is specified as pelitorine, and the deacidifying agents are selected from hydrotalcite, calcium carbonate, magnesium hydroxide, magnesium oxide, magnesium carbonate, sodium bicarbonate, or mixtures thereof. This patent claims a solid palatable composition intended for treating or preventing gastric disorders or diseases such as acid indigestion, heartburn, or gastritis, including at least one acid neutralizer and a neutral salivation stimulant.

Finally, the patent U.S. Pat. No. 6,746,697B2, entitled “Compositions containing Heliopsis longipes root extract and an oral vehicle,” is based on Heliopsis longipes root and used orally to treat xerostomia, dry mouth, to increase salivation, to relieve mouth dryness, and to maintain or improve oral hygiene. It also acts as a local anesthetic, cough suppressant, and as an oral product that produces desirable sensations in the oropharynx.

The herbal composition includes a Heliopsis longipes root extract and an oral vehicle for the extract. An adequate amount of extract ranges from approximately 0.01% to 10% by weight of the total composition. The preferred compositions include: (1) chewing gum with a solid exterior and liquid center, both containing an effective amount of extract composition; (2) solid gum; (3) lozenges and candies; and (4) flavored liquid sprays, drops, and mouth rinses. The invention is also useful as a flavor enhancer and taste booster.

All of the above patents disclose the use of the plant for technical effects that are traditionally known but only incipiently documented regarding the species in question.

Some online publications also anticipate the use of this plant in connection with alcoholic beverages:

“Gin, whiskey, and corn liquor—the Mexican spirits you must try.”

Domestic distilled spirits are gaining ground against major international brands. Among these, a family tradition led Mariana Torres Landa and her team to develop this distilled spirit that includes the chilcuague root in its formula:

“My grandfather always served it at home for his guests; he passed it down to the next generations. My father used to offer it to us on special occasions, and as the third generation, we decided to commercialize it. We made several adjustments to the flavor, the clarity, and the formula so that everyone who tries it can enjoy it. We've been working on commercialization for about seven years, and finally launched it in March 2020,” she explains.

The presentation came at a difficult time, but little by little this project—rooted in an ancestral and endemic plant from the Sierra Gorda, known as the golden root—has been gaining recognition:

“Chilcuague is the commercial name. We registered it before the Mexican Institute of Industrial Property (IMPI), and that's how we decided to name the product: Chilcuague, a distilled spirit made from blue agave (Agave tequilana Weber) with an artisanal maceration of the root, resulting in citrus aromas with herbal and raw agave notes.”

Indeed, there are several trademark registrations that differ from the formulations themselves; however, these do not affect the alcohol concentration or the preparation based on plant-derived ferments with functional advantages.

The present invention discloses a composition of Heliopsis longipes macerated in an alcoholic preparation for producing high-consumption alcoholic beverages such as tequila, rum, and charanda (a sugarcane distillate), among others. This process not only creates a synergistic effect between the plant's properties and the maceration process but also enhances the functional and sensory profile of the resulting beverage.

### DESCRIPTION OF THE DRAWINGS

The characteristic details of this novel composition for the production of alcoholic beverages based on Heliopsis longipes and its manufacturing process are clearly shown in the following description and the accompanying figures, where the same reference signs are used to indicate the parts and figures illustrated.

FIG. 1 is a general representative diagram of the morphology of the chilcuague plant and its features, highlighting: A) Root, B) Flower head, C) Peduncle, D) Ligulate flower, E) Disc flower, F) Achene of the ligulate flower, G) Achene of the disc flower, H) Stamens, I) Leaf.

FIG. 2 is a close-up and cross-section view of the hydrated chilcuague root (a) and the tears (b) caused by friction.

FIG. 3 is a close-up photograph of the bottle neck, showing the presence of white mold (cottony texture), a phenomenon of fungal contamination (May 2023).

FIG. 4 is a photograph of a bottle sectioned at the neck, showing the presence of black spots on the plant—a phenomenon of fermentation and fungal growth due to water presence (May 2023).

FIG. 5 is a photograph of a bottle sectioned at the neck, showing white mold proliferated in dispersed spots along the root (July 2023).

FIG. 6 is a photograph of a bottle sectioned at the neck, showing the presence of white mold (July 2023).

FIG. 7 is a photograph of a bottle sectioned at the neck, showing white mold on the roots with a cottony texture, indicating a possible humidity issue (July 2023).

FIG. 8 is a diagram of the process for the preparation of chilcuague.

FIG. 9 is a view showing a container where the internal pressure generated can be observed.

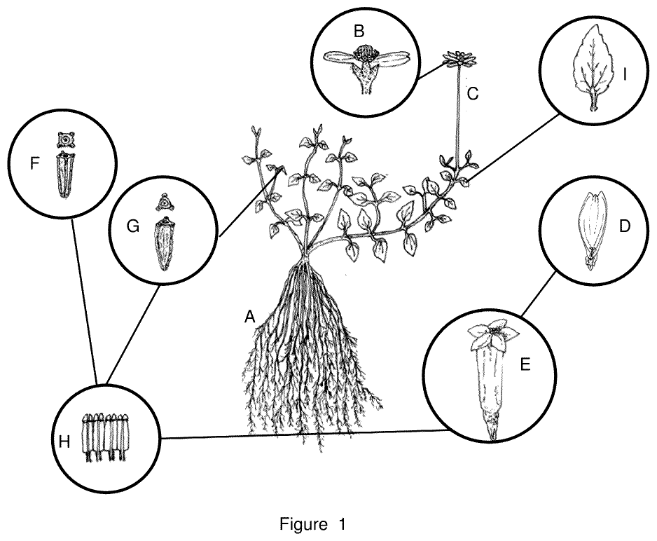

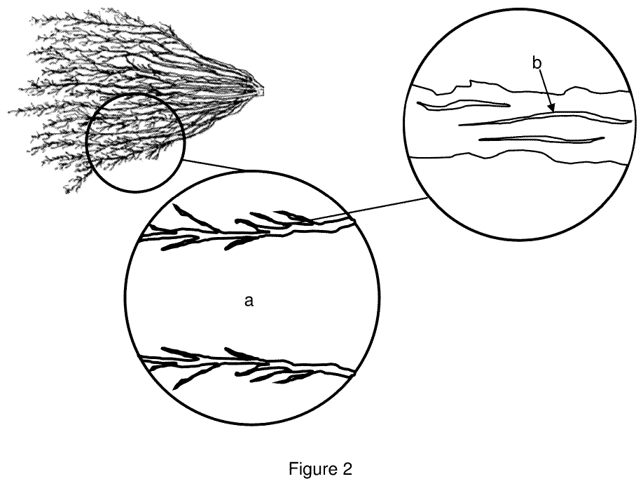

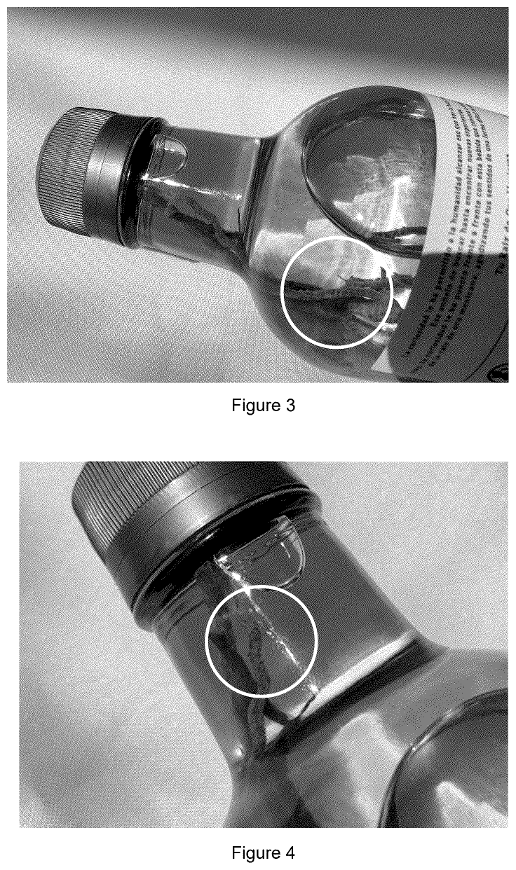

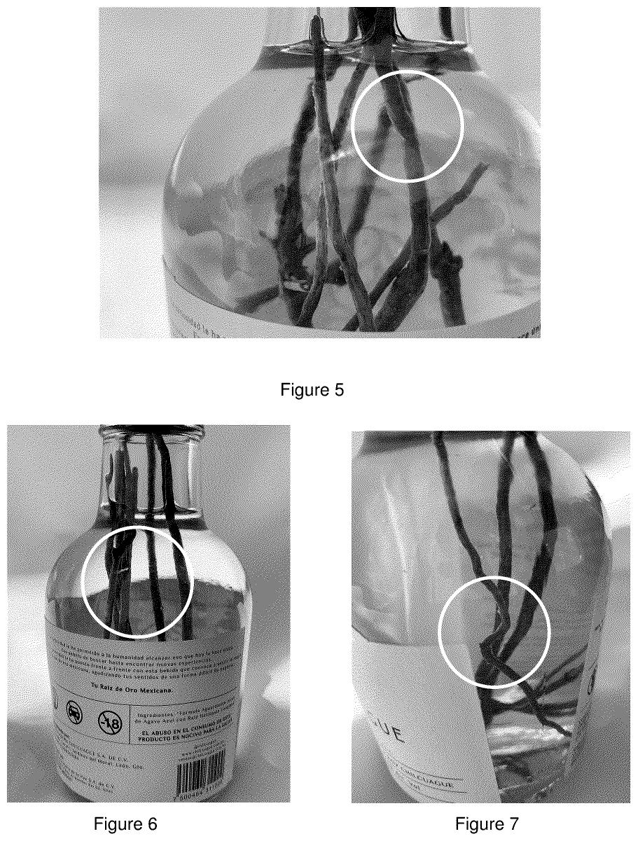

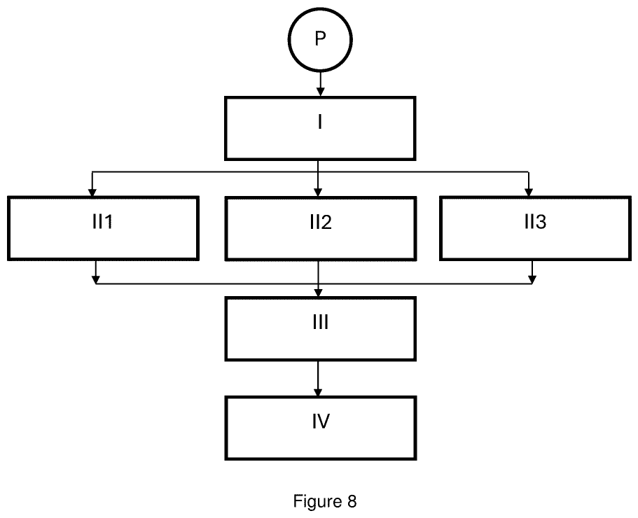

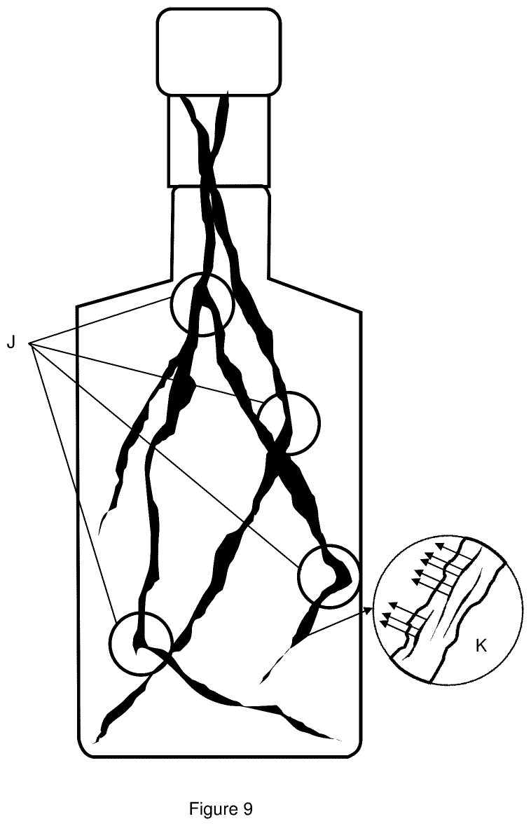

### DESCRIPTION

Heliopsis is a genus belonging to the tribe Heliantheae of the Asteraceae family and is distinguished by having both ligulate and disc flowers that are hermaphroditic and fertile. The ligules are persistent and sessile; the achenes of both flower types are thick, tri- or tetrangular, with the pappus absent or greatly reduced. The leaf margins are generally serrated or crenate, and the involucre is not distinctly graduated.

The genus is native to the Americas, with 32 known species (International Plant Name Index). Of these, eleven are endemic to Mexico (H. anomala B. L. Turner, H. annua Hemsl., H. brachactis Standl. ex Fisher, H. filifolia S. Watson, H. longipes S. F. Blake, H. novogaliciana B. L. Turner, H. parviceps S. F. Blake, H. procumbens Hemsl., H. rubra Fisher, H. sinaloensis B. L. Turner, and H. sufruticosa Ramírez-Noya et S. González). Two others, H. buphthalmoides Dun. and H. parvifolia A. Gray, are distributed from Mexico to South America.

Heliopsis longipes is a microendemic species of the Sierra de Álvarez and Sierra Gorda regions in the states of San Luis Potosi and Guanajuato; it is the most economically important species of its genus. Its root has traditional uses as a condiment, medicine, and insecticide. From the root of H. longipes, an alkamide known as affinin was isolated, to which the plant's well-known insecticidal properties are attributed.

After a learning process spanning over ten years, the following process has been deduced, based on observation, experimentation, and scientific methods used to analyze samples by various techniques until establishing the process described below.

Based on the figures described above, the composition comprises the following process:

In different cultivation sites, the root of Heliopsis longipes grows underground and generally develops as robust, capillary-like roots. These are unearthed and rinsed with water to remove excess minerals, contaminants, and soil. The bundles are then sent to a collection center where the method begins:

### 1. Cleaning by Immersion

The bark of the Heliopsis longipes root is rough, dry, and coarse, and completely dehydrated at the time of cutting. This stage must remove impurities from the plant. The roots are cut to a length between 1 and 2 cm longer than the container in which they will be macerated, so that they slightly protrude from it. After cutting, the cleaning stage transitions into hydration.

### 2. Hydration by Immersion

The roots are immersed in a container filled with water at a temperature between 30° C. and 37° C. for at least one hour, during which the Heliopsis longipes roots must remain completely submerged.

### 3. Cutting by Immersion

After being submerged for at least one additional hour, the roots are aligned to form bundles weighing approximately 10 to 40 grams of biomass. The ends are cut so that the bundles have a length approximately one or two centimeters greater than the container in which they will later be placed to release their organoleptic properties-important for the final technical effect.

During this stage, the formation of bundles must also occur while submerged, to avoid oxidation of the Heliopsis longipes.

The immersion can be performed using softened water under any of the following conditions:
II1. To remove temporary hardness (carbonate hardness), the water is boiled, and for each liter of water, between 10 and 39 grams of calcium hydroxide (Ca(OH)₂) are added. The Heliopsis longipes root is kept submerged for at least one hour.
II2. Using demineralized water or water filtered by conventional means such as reverse osmosis (RO), activated carbon, ceramics, or zeolite, to reduce the mineral content and water hardness, achieving a purity of at least 50% per liter.
II3. Using pure water enriched with calcium salts to create “hard water” (as opposed to “soft water”) containing high levels of minerals—particularly magnesium and calcium salts. Preferably, water with hardness above 120 mg/L CaCO₃ is used. In this case, water is boiled to soften it; upon reaching the boiling point, the boiling is maintained for at least five minutes until CaCO₃ sediment forms. After two to three hours, the CaCO₃ precipitates and the water is ready.

### 4. Bark Tearing by Immersion

The Heliopsis longipes roots are kept completely submerged in water. When the roots exhibit overhydration and irregular bulging from excess water, they are rubbed to produce tearing of the outer bark. This process facilitates the release of lactic acid, affinin, and lignin due to molecular disruption of the root structure.

Since water, heat, and time can promote the decomposition of lactic acid, affinin, and lignin, the bundles are removed from the water and dried using a porous material, applying pressure to eliminate excess moisture. The roots are then pressed to close the tears caused by friction during this stage.

### 5. Maceration

The bundles of Heliopsis longipes are macerated in an alcohol concentration ranging from 10% to 45%, preferably at 35%. The roots must be completely dry, as trapped moisture could accelerate decomposition, causing changes in color, texture, and flavor, as well as loss of properties due to oxidation of lactic acid, affinin, and lignin.

If humidity accumulates and air circulation is insufficient, mold and bacteria can develop.

During this stage, the roots are cut into pieces approximately 1-2 cm longer than the container, protruding slightly from it. They are placed in a solvent containing at least 35% alcohol, with H. longipes in an approximate ratio of 0.5 to 10 grams of root per container, for a period between 15 and 45 days at temperatures between 25° C. and 35° C. After this period, the alcoholic extract is separated and stored.

### 6. Preservation

The extract obtained in the previous step is mixed with an alcoholic vector in a proportion between 10% and 45% alcohol, preferably at 35%. The alcoholic vector may be derived from plant-based spirits such as tequila, rum, vodka, gin, cachaça, or pisco, which typically have an alcohol content exceeding 30% v/v ethanol.

The roots must remain immersed to prevent mold growth; however, it is important that the root stays bent inside the container so that the bark tears produced in the immersion stages reopen and rehydrate in the alcoholic vector, allowing it to absorb the organoleptic characteristics of Heliopsis longipes.

For this reason, when placing the bundle in the container, pressure must be applied to keep the root folded and the bark ruptures open—this is achieved by the root length specified in the first step. When the cap of the container is closed, it presses the Heliopsis longipes roots, keeping them submerged until the liquid is fully consumed.

It is important to note that the extraction of Heliopsis longipes transfers to the alcoholic vector specific organoleptic properties, such as:
A highly astringent flavor;
A light honey-colored tone (which depends on how long the root remains submerged in the alcoholic vector);
A neutral aroma relative to the vector—that is, the vector provides the alcoholic scent, while Heliopsis longipes does not impart any distinctive odor;
A strongly astringent taste that stimulates the taste buds and salivation, enhancing the organoleptic perception of any food consumed concurrently.

The root of Heliopsis longipes in the alcoholic vector causes accelerated decomposition in the presence of air mixed with the alcoholic solvent.

When water is mixed into the alcoholic vector, fungal growth accelerates, leading to the degradation of the alcoholic mixture.

The lactic acid present inhibits hangover effects from consumption of the alcoholic vector.

The vector also acquires healing, emollient, and moisturizing properties.

When Heliopsis longipes is placed in water alone, it causes dryness of the skin in humans.

Through this process, a composition has been obtained that can be introduced into the production chain of alcoholic beverages without violating the standards governing their denomination of origin, as shown in the following examples.

Additionally, the alcohol and its combination with this process allow the generation of lactic acid, giving the alcoholic beverage a technical advantage by increasing the production of this compound. Moreover, the beverage produced through this process improves the organoleptic, astringent, and sensory experience.

Lactic acid is commonly produced in various biological processes—such as muscle exertion—and in the biotechnological industry it is obtained by lactic fermentation of sugars from milk (lactose), grapes (glucose), or sugarcane (sucrose), as well as from starch, sour milk, and other derivatives like cottage cheese and yogurt.

However, it has been scarcely studied in roots such as chilcuague. In this invention, the ability to enhance the organoleptic experience of alcoholic beverages makes it possible to generate lactic acid directly from the root through an exclusive process of alcoholic maceration balanced with a diluent as described herein.

In the alcoholic beverage industry—tequila, rum, vodka, gin, cachaça, pisco, among others—all these beverages share the characteristic of having an alcohol content above 30% v/v ethanol. Although they are obtained through fractional distillation of fermented liquids, none have incorporated lactic acid generation within their process as a flavor enhancer, nor has any beverage used an endemic root to achieve it. This invention thus provides a process compliant with technical standards for such beverages, as seen in the Mexican Official Standard NOM-006-SCFI-2012, which establishes the technical specifications for tequila. The standard presents the following table:

TABLE 1
PHYSICOCHEMICAL SPECIFICATIONS OF TEQUILA

| | | Tequila | | | Tequila |
|---|---|---|---|---|---|
| | Tequila | Joven | Tequila | Tequila | Extra |
| Parameter | Blanco | or Oro | Reposado | Añejo | Añejo |
| Alcohol Content at 293 K (20° C.) | 35-55 | 35-55 | 35-55 | 35-55 | 35-55 |
| (% Alc. Vol.) | | | | | |
| Dry Extract (g/L) | 0-0.30 | 0-5 | 0-5 | 0-5 | 0-5 |
| Higher Alcohols (alcohols with | 20-500 | 20-500 | 20-500 | 20-500 | 20-500 |
| molecular weight greater than | | | | | |
| ethanol, e.g., isoamyl alcohol) | | | | | |
| (mg/100 mL anhydrous alcohol) | | | | | |
| Methanol (mg/100 mL anhydrous | 30-300 | 30-300 | 30-300 | 30-300 | 30-300 |
| alcohol) | | | | | |
| Aldehydes (as acetaldehyde) | 0-40 | 0-40 | 0-40 | 0-40 | 0-40 |
| (mg/100 mL anhydrous alcohol) | | | | | |
| Esters (as ethyl acetate) (mg/100 | 2-200 | 2-200 | 2-250 | 2-250 | 2-250 |
| mL anhydrous alcohol) | | | | | |
| Furfural (mg/100 mL anhydrous | 0-4 | 0-4 | 0-4 | 0-4 | 0-4 |
| alcohol) | | | | | |

### Notes:

(1) See Chapter 3, References.
(2) The minimum parameter may be reduced if the tequila producer demonstrates to the Conformity Assessment Body that a viable method is available to reduce methanol content.
(3) For the determination of higher alcohols under this standard, only the Gas Chromatography method is permitted, since the spectrophotometric (wet) method does not quantify the n-propanol present in samples. This restriction prevents significant discrepancies in the measurement of higher alcohols depending on whether the chromatographic or wet method is used in the laboratory.

In accordance with this standard, maceration is permitted below 1%, as established by law. However, due to this maceration, the presence of lactic acid can be increased, providing a unique advantage in the organoleptic sensation of the resulting alcoholic beverage.

### Example 1—Tequila

Once the maceration was obtained, tests were performed on two bottles from different batches and years of the composition in an alcoholic beverage. From each original 750 ml bottle, 200 mL were extracted and sent to the laboratory-meaning only the pre-macerated distillate was analyzed. Each bottle was identified as follows:
Bottle #1
Bottled: Oct. 19, 2023
Batch: 11
Sampled: May 3, 2024
Bottle #2
Bottled: Feb. 1, 2024
Batch: 13
Sampled: May 9, 2024

Subsequently, it was determined that the maceration contained the following composition, shown in Table 2.

TABLE 2
Composition of the maceration prior
to mixing with the distilled beverage

| Component | Bottle #1 | Bottle #2 |
|---|---|---|
| Methyl alcohol | 0.26 | 0.29 |
| Ethyl alcohol | 100 | 100 |
| 1-propanol | 0.58 | 0.54 |
| Ethoxyacetic acid | — | 0.34 |
| Ethyl acetate | 0.06 | 0.10 |
| 1-nitropropane | — | 0.07 |
| 2-butanol | 0.03 | — |
| 2-methyl-1-propanol | 0.09 | 0.09 |
| Acetic acid | 0.11 | 0.13 |
| 3-methyl-1-butanol | 0.17 | 0.23 |
| 2-methyl-1-butanol | 0.04 | 0.05 |
| Ethyl lactate | 0.02 | 0.02 |
| 1-phenylethanol | 0.004 | 0.004 |

The following components may be classified according to the compound type specified in NOM-006-SCFI-2012, Alcoholic Beverages—Tequila—Specifications:
Higher alcohols (alcohols of molecular weight greater than ethanol): 1-propanol, 3-methyl-1-butanol, 2-methyl-1-butanol, 2-methyl-1-propanol, 2-butanol, and 1-phenylethanol.
Methanol
Esters: ethyl lactate.
Organic acids: acetic acid, ethoxyacetic acid.
Other detected compounds: 1-nitropropane.

It is important to note that in this composition lactic acid was not detected.

After 30 days of maceration, new tests were conducted, and the following observations were made:

Again, tests were performed on two bottles from different batches and years. From each original 750 ml bottle, 200 mL were extracted for laboratory analysis-only the previously macerated distillate was sent, that is, after waiting the required process period.
Bottle #1
Bottled: Oct. 19, 2023
Batch: 11
Bottle #2
Bottled: Feb. 1, 2024
Batch: 13

Subsequently, it was determined that the maceration contained the following composition, shown in Table 3.

TABLE 3
Composition of the maceration prior to mixing with
the distilled beverage after maceration period

| Component | Bottle #1 | Bottle #2 |
|---|---|---|
| Methyl alcohol | 0.171 | 0.199 |
| Ethyl alcohol | 98.544 | 98.543 |
| Isopropyl alcohol | 0.004 | 0.005 |
| 1-propanol | 0.596 | 0.567 |
| Ethyl acetate | 0.064 | 0.118 |
| 2-butanol | 0.042 | 0.014 |
| 2-methyl-1-propanol | 0.144 | 0.132 |
| Acetic acid | 0.086 | 0.098 |
| 1,1-diethoxyethane | 0.006 | 0.007 |
| 3-methyl-1-butanol | 0.202 | 0.204 |
| 2-methyl-1-butanol | 0.073 | 0.081 |
| 2-hydroxypropanoic acid (lactic acid) | 0.037 | 0.029 |
| Furfural | 0.005 | 0.002 |
| 5-methyl-2-furancarboxaldehyde | 0.003 | 0.002 |
| Phenylethyl alcohol | 0.005 | 0.004 |

The following components may be classified according to the compound type specified in NOM-006-SCFI-2012, Alcoholic Beverages—Tequila—Specifications:
Higher alcohols: isopropyl alcohol, 1-propanol, 3-methyl-1-butanol, 2-methyl-1-butanol, 2-methyl-1-propanol, 2-butanol, phenylethyl alcohol.
Methanol
Esters: ethyl acetate.
Organic acids: acetic acid, lactic acid.
Aldehydes: furfural, 1,1-diethoxyethane.
Other detected compounds: 5-methyl-2-furancarboxaldehyde.

Note: The initial tests were performed on bottles whose samples were collected 30 days earlier. In those, the distillate was isolated before maceration had fully matured. In the second analysis, lactic acid was again detected, although in smaller amounts. The handling and storage conditions of the bottles likely influenced this outcome; the samples had been previously opened one month before, so the product was no longer new.

By contrast, tests conducted the previous year used brand-new, unopened bottles with no prior manipulation.

Furthermore, in the composition of Chilcuague, lactic acid is observed as a compound that develops under this process, as demonstrated by laboratory studies summarized below.

TABLE 4
Lactic acid present in Chilcuague root

| Component | Bottle #1 | Bottle #2 |
|---|---|---|
| 2-butanol | 0.94 | 0.59 |
| 2-hydroxypropanoic acid | 0.78 | 0.51 |
| Ethyl acetate | 0.58 | 0.73 |

| | |
|---|---|
| Range: | 0.51-0.78 |

TABLE 5
Lactic acid in Chilcuague bottle - first analysis

| Component | Concentration |
|---|---|
| 2-hydroxypropanoic acid (lactic acid) | 0.037 |

TABLE 6
Lactic acid in Chilcuague bottle - second analysis

| Component | Concentration |
|---|---|
| 2-hydroxypropanoic acid (lactic acid) | 0.039 |

The measured range of lactic acid in Chilcuague thus varies between 0.029% and 0.78%.

Having sufficiently described my invention, which I consider novel, I therefore claim exclusive ownership of the subject matter contained in the following claims:

1. A process for obtaining a composition for the preparation of alcoholic beverages based on Heliopsis longipes plant, the process comprising the following stages:
cleaning by immersion;
hydration by immersion;
cutting by immersion;
bark tearing by immersion;
maceration; and
preservation;
Wherein:
the step of cleaning by immersion includes: removing impurities from the plant and cutting roots to a length between 1 and 2 cm longer than a container where it will be macerated;
the step of hydration by immersion includes immersing the root in water at a temperature between 30° C. and 37° C. for at least one hour, ensuring that the Heliopsis longipes root remains completely submerged;
the step of cutting by immersion includes:
aligning the plant to form bundles weighing approximately 10 to 40 grams of biomass, wherein the bundles are trimmed so that the bundles measure about one or two centimeters longer than the container in which they will be placed to release their organoleptic, wherein the bundles are formed underwater to avoid oxidation of Heliopsis longipes, wherein the Immersion is carried out using softened water under any of the following conditions:
II1. temporary (carbonate) hardness is removed by boiling the water and adding between 10 and 39 grams of calcium hydroxide (Ca(OH)₂) per liter, keeping the Heliopsis longipes root submerged for at least one hour;
II2. using demineralized water or water filtered by reverse osmosis (RO), activated carbon, ceramic, or zeolite filters to reduce mineral content and water hardness by at least 50% per liter;
II3. using pure water adjusted with calcium salts to produce hard water—defined as water with a high content of minerals, particularly magnesium and calcium salts, wherein the water has a hardness greater than 120 mg/L CaCO₃ and is prepared by boiling the water, maintaining the boil for at least five minutes, and allowing calcium carbonate sediment to form, after 2 to 3 hours, the CaCO₃ settles and the water is decanted;
the step of bark tearing by immersion includes keeping the Heliopsis longipes roots a fully submerged, when the roots show overhydration and irregular swelling from excess water, the roots are rubbed to induce tearing of the outer bark, the overhydration facilitates the release of lactic acid, affinin, and lignin due to molecular disruption of the root, wherein because of the water, the heat, and the time promote decomposition of lactic acid, affinin, and lignin, the bundles are removed from the water, dried with a porous material, and pressed to eliminate excess moisture, pressure is applied to close the bark tears caused by friction during this step;
the maceration includes: macerating the Heliopsis longipes bundles in an alcoholic solution with a concentration between 10% and 45%, the roots must be completely dry, as trapped moisture may accelerate decomposition, causing changes in color, texture, and flavor, as well as the loss of properties through oxidation of lactic acid, affinin, and lignin, the roots are cut into pieces about 1-2 cm longer than the container so that they slightly protrude, and are placed in a solvent with at least 35% alcohol by volume, in a proportion of about 0.5 to 10 grams of root per container, for 15 to 45 days at 25-35° C., then the alcoholic extract is separated and stored; and
the step of Preservation includes: mixing the extract obtained from the previous step with an alcoholic vector in a concentration between 10% and 45% alcohol; wherein the alcoholic vector is a plant-based spirit such as tequila, rum, vodka, gin, cachaça, or pisco, with an ethanol concentration above 30% v/v, the root must remain immersed to prevent mold proliferation, inside the container, the root must remain bent so that the tears formed during previous steps reopen and rehydrate with the alcoholic vector, which absorbs the organoleptic properties of Heliopsis longipes, then the bundle is inserted under pressure to keep the root folded and the bark openings exposed.

2. A composition obtained by the process of claim 1 comprising:
the Heliopsis longipes macerated extract prepared in an alcohol concentration between 10% and 45%, preferably 35%, wherein the roots of the plant are completely dry wherein the roots are placed in a solvent with at least 35% alcohol and H. longipes in a proportion of 0.5 to 10 grams per container, for 15-45 days at 25-35° C., then the alcoholic extract is separated and stored.

3. The composition of claim 2, wherein the alcoholic vector is a beverage selected from tequila, rum, vodka, gin, cachaça, or pisco—all of which have an ethanol concentration above 30% v/v—and which acquire antiseptic, astringent, deworming, and organoleptic properties from the lactic acid present in the alcoholic vector.

4. The composition of claim 2, wherein the alcoholic vector is used in the manufacture of cosmetics, food concentrates such as kefir, whey, yogurt, sauces, marinades, sweets, lemonades, soft drinks, jams, or preserves.

5. The composition of claim 2, wherein the alcoholic vector is used in the production of cleaning agents for various surfaces, as well as shampoos, creams, gels, ointments, and antibacterial, anti-inflammatory, and antipruritic medications, pharmaceuticals, dietary supplements, beekeeping solutions for controlling mites in hives, or animal feed.

6. The composition of claim 2, wherein the alcoholic vector is used as a natural tanning agent, given that lactic acid is also used in the tanning of hides, or the treatment of textiles.
